(19) United States
(12) Patent Application Publication
(10) Pub. No.: US 2025/0069745 A1
(43) Pub. Date: Feb. 27, 2025

(54) DIAGNOSIS SUPPORT DEVICE, RECORDING MEDIUM, AND DIAGNOSIS SUPPORT METHOD

(71) Applicant: DAI NIPPON PRINTING CO., LTD., Tokyo (JP)

(72) Inventors: Tetsutaro ONO, Tokyo (JP); Takahiro TANAKA, Tokyo (JP)

(73) Assignee: DAI NIPPON PRINTING CO., LTD., Tokyo (JP)

(21) Appl. No.: 18/574,190

(22) PCT Filed: Jun. 6, 2022
(86) PCT No.: PCT/JP2022/022750

§ 371 Date: May 21, 2024

(30) Foreign Application Priority Data
Jun. 29, 2021 (JP) 2021-107793

(51) Int. Cl. G16H 50/20; A61B 5/00; G06T 7/00

(52) U.S. Cl. CPC G16H 50/20; A61B 5/0042; G06T 7/0012; G06T 2207/30016

(57) ABSTRACT

A diagnosis support device, a recording medium, and a diagnosis support method that can support a doctor's diagnosis. The diagnosis support device includes: an acquisition unit acquiring subject data related to a brain of a subject; a prediction unit predicting a brain disease of the subject on the basis of the subject data; a specification unit specifying a data item corresponding to subject data, which contributes to a prediction result, in the subject data; and an output unit outputting the specified data item and prior knowledge related to the brain disease in association with each other.

### REFERENCE TO RELATED APPLICATIONS

This application is the national phase under 35 U.S.C. § 371 of PCT International Application No. PCT/JP2022/022750 which has an International filing date of Jun. 6, 2022 and designated the United States of America.

The present disclosure relates to a diagnosis support device, a recording medium, and a diagnosis support method.

### BACKGROUND ART

For brain diseases including Alzheimer's dementia and the like, it is effective to perform appropriate treatment on the brain disease at an early stage of the disease. Therefore, an early diagnosis is required. In a field of computer-aided diagnosis (CAD), there is a technique in which a diagnosis system performs analysis on the basis of images and clinical information of a patient. A doctor is expected to be able to make a more accurate diagnosis with reference to the results output by the diagnosis system as a second opinion.

In recent years, diagnosis systems using machine learning have also been devised. International Publication No. 2021/020198 discloses an artificial intelligence technique that predicts an aspect related to a matter at a time different from an imaging time on the basis of a combination of image data and non-image data.

### SUMMARY

However, in the diagnosis system using the artificial intelligence technique, internal processing is complicated, and an intermediate determination process and a prediction algorithm are concealed.

Therefore, even when a diagnosis result is output, the basis for the diagnosis result may be unclear, and it may be difficult for the doctor to refer to the basis when the doctor makes a diagnosis.

The present disclosure has been made in view of the above circumstances, and an object of the present disclosure is to provide a diagnosis support device, a recording medium, and a diagnosis support method that can support a doctor's diagnosis.

The present application includes a plurality of means for achieving the object and provides, as an example thereof, a diagnosis support device including: an acquisition unit acquiring subject data related to a brain of a subject; a prediction unit predicting a brain disease of the subject on the basis of the subject data; a specification unit specifying a data item corresponding to subject data, which contributes to a prediction result of the prediction unit, in the subject data; and an output unit outputting the data item specified by the specification unit and prior knowledge related to the brain disease in association with each other.

### DESCRIPTION

According to the present disclosure, the basis for predicting a brain disease and prior knowledge are associated with each other. Therefore, a doctor can refer to the basis when making a diagnosis, and it is possible to support the doctor's diagnosis.

The above and further objects and features of the invention will more fully be apparent from the following detailed description with accompanying drawings.

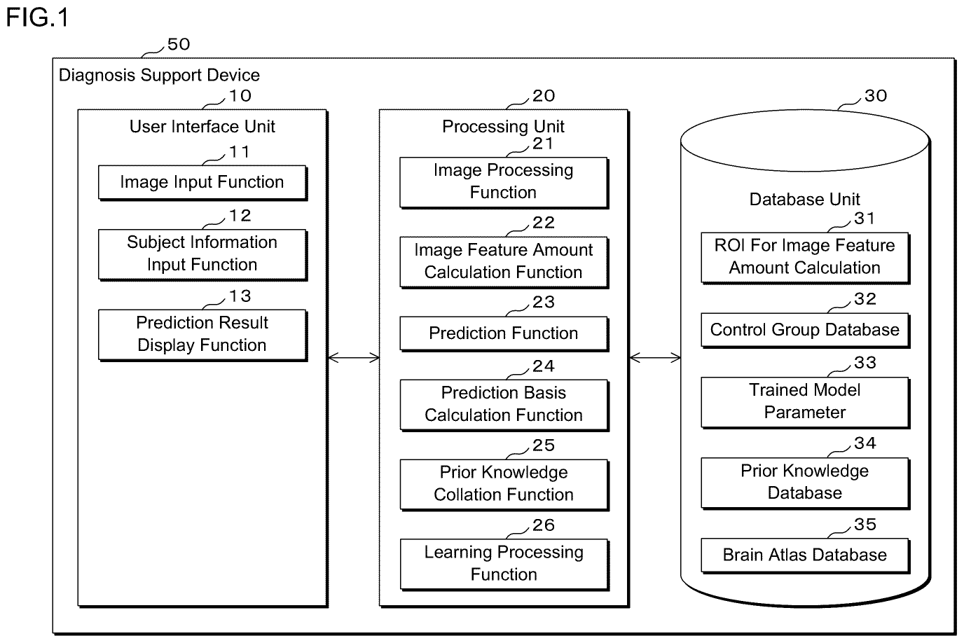

FIG. 1 is a diagram illustrating an example of a configuration of a diagnosis support device according to this embodiment.

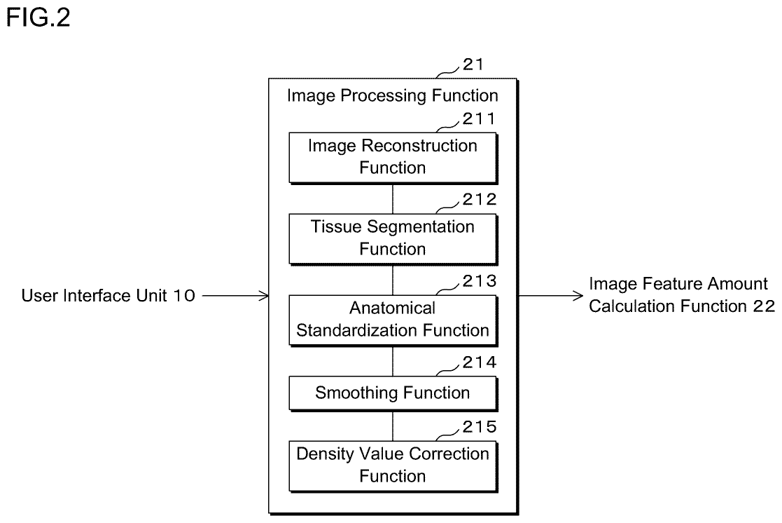

FIG. 2 is a diagram illustrating an example of a configuration of an image processing function.

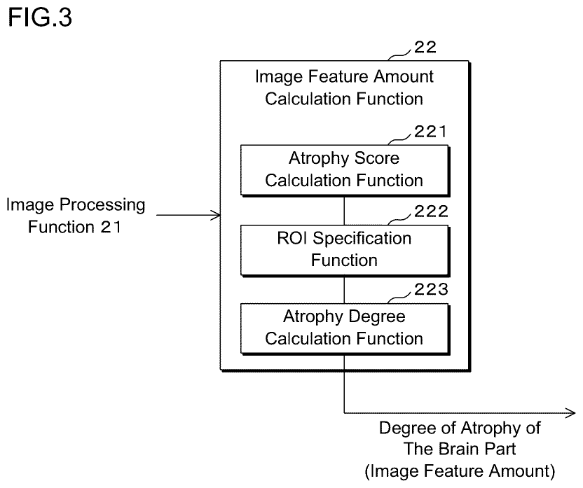

FIG. 3 is a diagram illustrating an example of a configuration of an image feature amount calculation function according to a first embodiment.

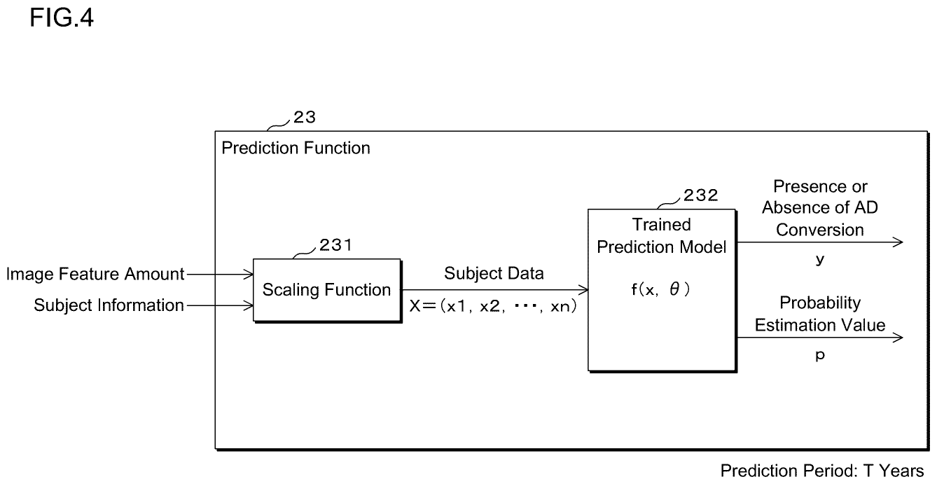

FIG. 4 is a diagram illustrating an example of a configuration of a prediction function.

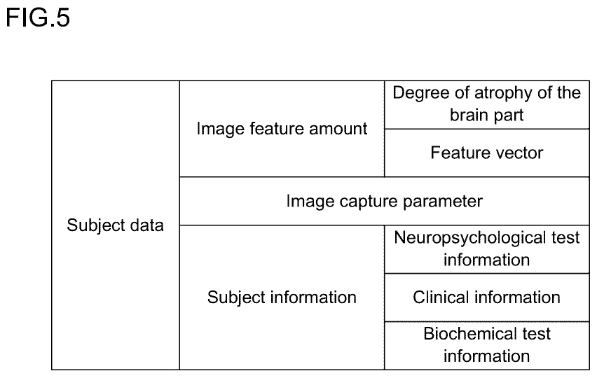

FIG. 5 is a diagram illustrating an example of a configuration of subject data.

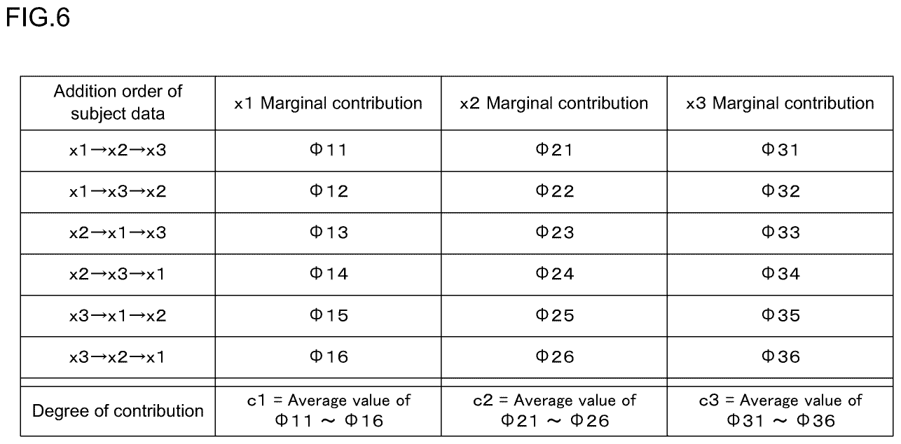

FIG. 6 is a diagram illustrating an example of calculation of a degree of contribution.

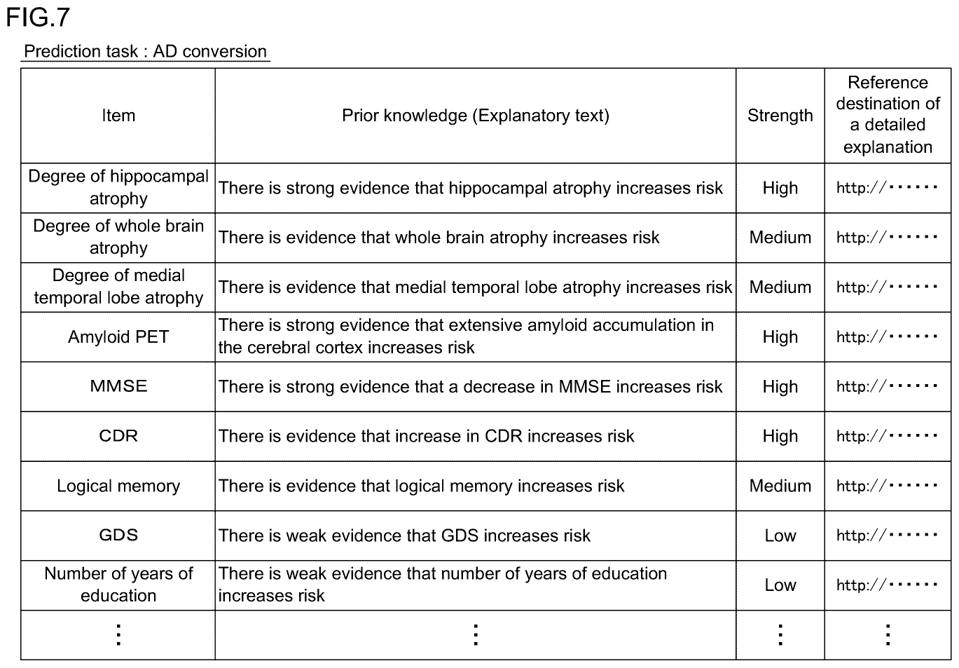

FIG. 7 is a diagram illustrating an example of a configuration of a prior knowledge database.

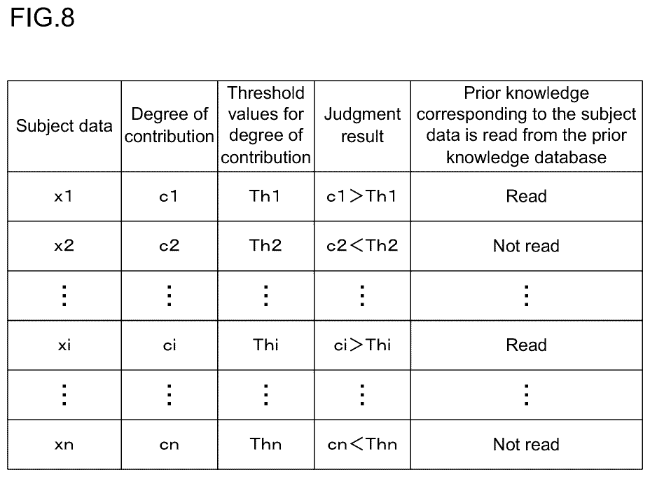

FIG. 8 is a diagram illustrating an example of a method for reading prior knowledge.

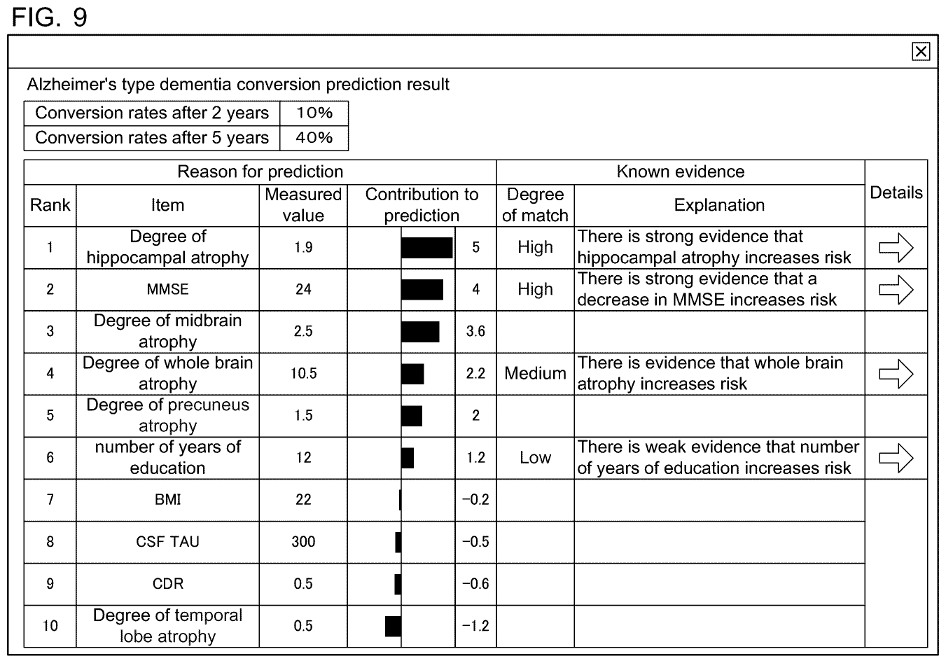

FIG. 9 is a diagram illustrating a first example of display of a prediction result.

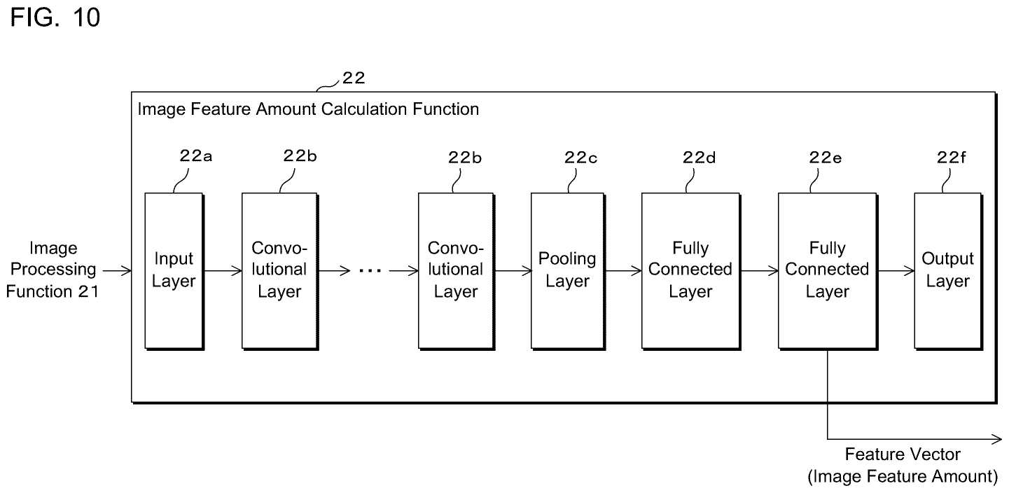

FIG. 10 is a diagram illustrating an example of a configuration of an image feature amount calculation function according to a second embodiment.

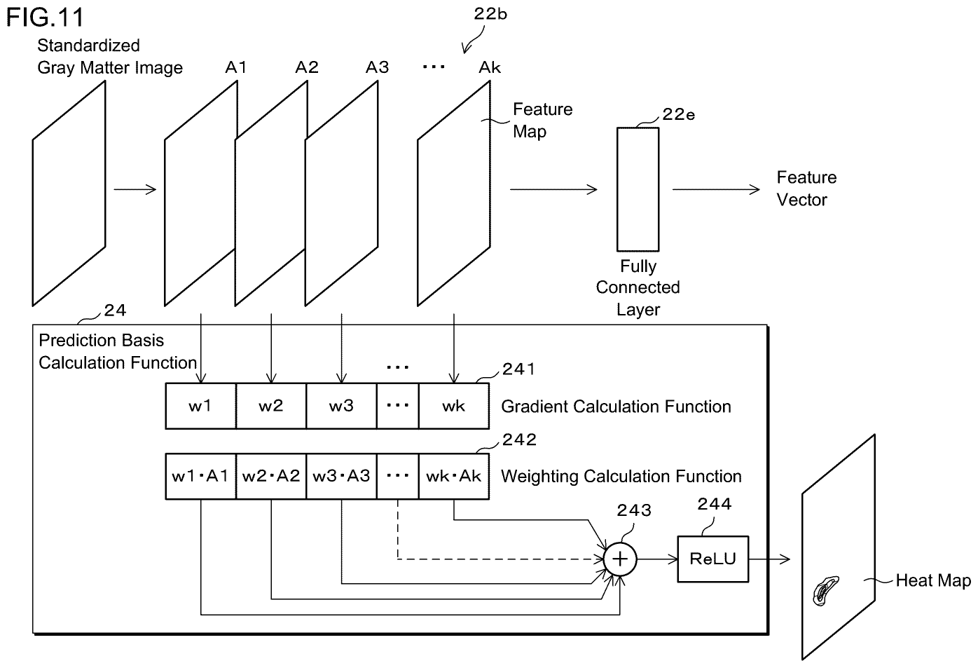

FIG. 11 is a diagram illustrating an example of a configuration of a prediction basis calculation function according to the second embodiment.

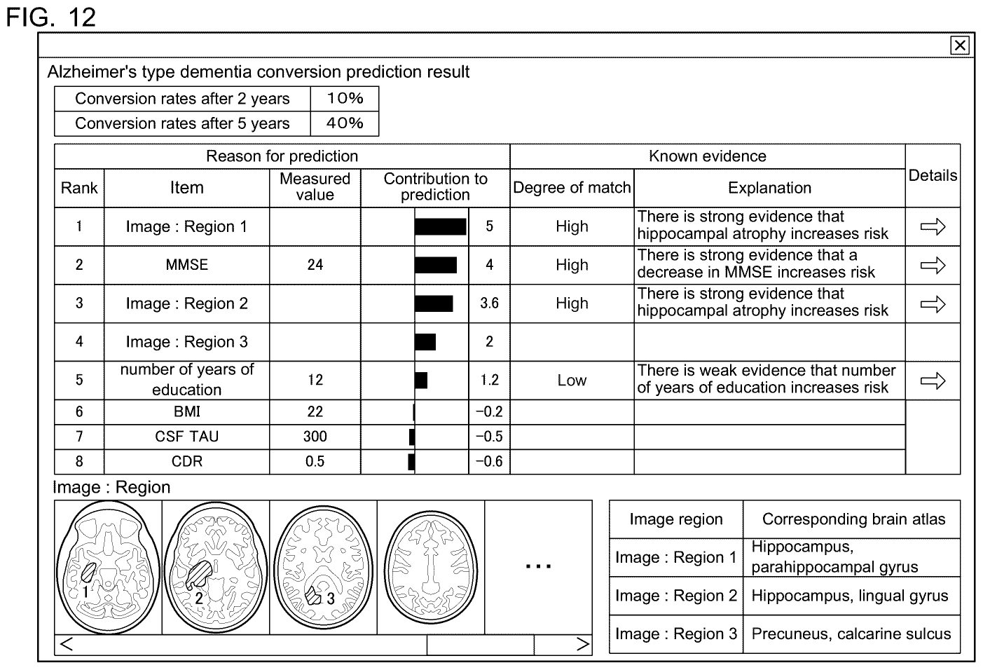

FIG. 12 is a diagram illustrating a second example of the display of the prediction result.

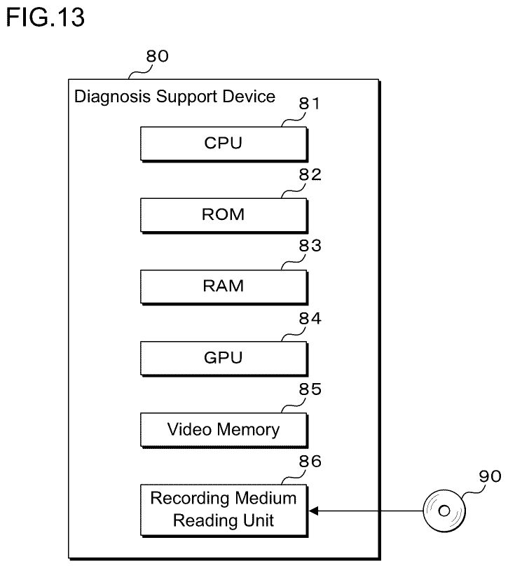

FIG. 13 is a diagram illustrating an example of a configuration of a diagnosis support device according to this embodiment.

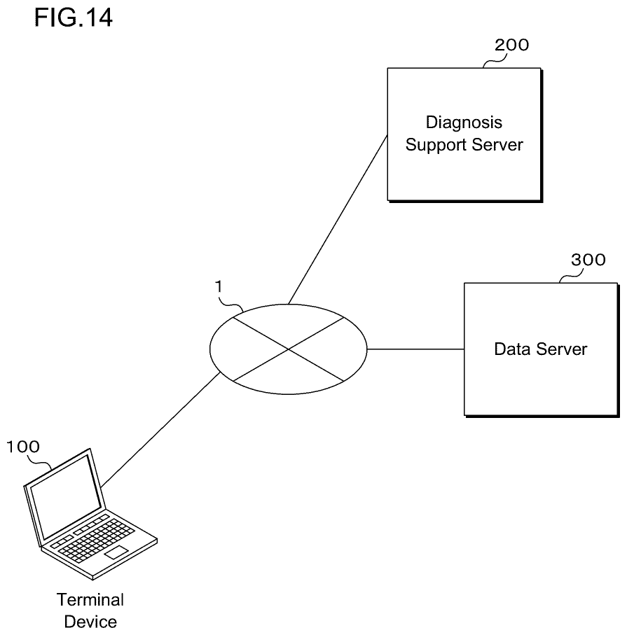

FIG. 14 is a diagram illustrating an example of a configuration of a diagnosis support system.

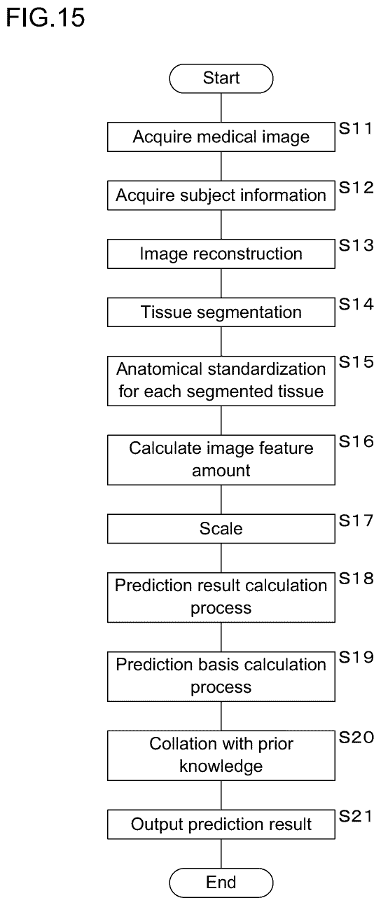

FIG. 15 is a diagram illustrating a procedure of a prediction process.

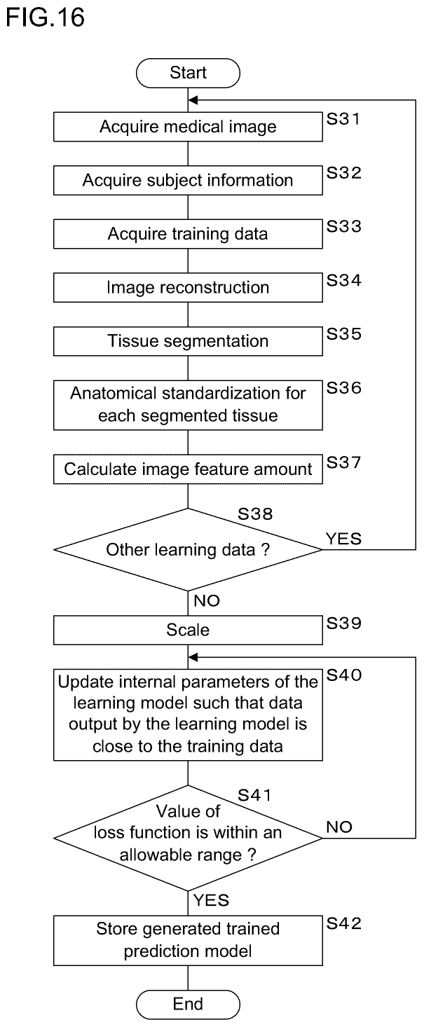

FIG. 16 is a diagram illustrating a procedure of a process of generating a trained prediction model.

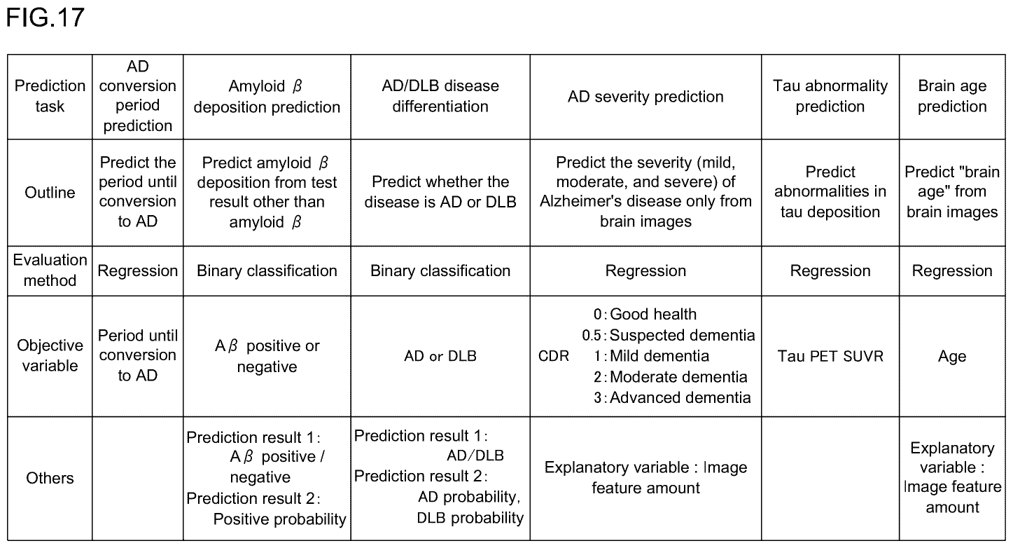

FIG. 17 is a diagram illustrating other prediction tasks.

### First Embodiment

Hereinafter, embodiments of the present invention will be described with reference to the drawings. FIG. 1 is a diagram illustrating an example of a configuration of a diagnosis support device 50 according to this embodiment. The diagnosis support device 50 includes a user interface unit 10, a processing unit 20, and a database unit 30. The user interface unit 10 includes an image input function 11, a subject information input function 12, and a prediction result display function 13. The processing unit 20 includes an image processing function 21, an image feature amount calculation function 22, a prediction function 23, a prediction basis calculation function 24, a prior knowledge collation function 25, and a learning processing function 26. The database unit 30 includes a region of interest (ROI) for image feature amount calculation, a control group database 32, a trained model parameter 33, a prior knowledge database 34, and a brain atlas database 35. The database unit 30 may be incorporated into the diagnosis support device 50. Alternatively, the database unit 30 may be provided outside the diagnosis support device 50 so as to be accessed by the diagnosis support device 50. The diagnosis support device 50 can output prediction results for a plurality of types of prediction tasks in order to support diagnosis related to the brain. Hereinafter, AD conversion will be described as an example of the prediction task. The AD conversion is for predicting the possibility that a person with a normal cognitive function or a person with mild cognitive impairment (MCI) will convert to Alzheimer's disease (AD) after a certain number of years (for example, 1 year, 2 years, 3 years, 5 years, 10 years, and the like).

The image input function 11 includes an interface function with a magnetic resonance imaging (MRI) apparatus or an image DB (not illustrated) and can acquire (receive) medical images related to the brain of a subject (including a patient). The medical image is, for example, an MRI image (also referred to as an MR image). However, the medical image is not limited to the MRI image. For example, the medical image may be a positron emission tomography (PET) image that can be acquired from a PET apparatus, a single photon emission CT (SPECT) image that can be acquired from a SPECT apparatus, or a computed tomography (CT) image that can be acquired from a CT apparatus. The MRI images include not only MRI images (for example, a T1-weighted image, a T2-weighted image, a diffusion-weighted image, and the like) obtained by the MRI apparatus, but also processed images obtained by performing predetermined calculations on MRI signals. Hereinafter, the MRI image will be described as an example of the medical image.

The subject information input function 12 includes a function of inputting subject information and can acquire (receive) subject information from an external device. The subject information will be described in detail below.

The prediction result display function includes a function of displaying the prediction result of the processing unit 20 on a display device (display unit) (not illustrated). The prediction result will be described in detail below.

FIG. 2 is a diagram illustrating an example of a configuration of the image processing function 21. The image processing function 21 includes an image reconstruction function 211, a tissue segmentation function 212, an anatomical standardization function 213, a smoothing function 214, and a density value correction function 215. In addition, a portion or all of the image processing function 21 may be omitted.

The image reconstruction function 211 performs image reconstruction on the MRI image of the subject acquired from the image input function 11. The image reconstruction converts the MRI image (three-dimensional image) of the subject into, for example, 100 to 200 T1-weighted images captured in slices with a predetermined thickness so as to include the entire brain. At this time, the slice images are resampled such that the lengths of the sides of voxels in each slice image are equal to each other in advance. Then, the MRI image of the subject is spatially registered with a standard brain image. Specifically, linear transformation (affine transformation), trimming, and the like are performed on the MRI image of the subject such that the position, angle, size, and the like of the MRI image are matched with those of the standard brain image. Therefore, for example, the deviation of the position of the subject's head when the MRI image is captured is corrected on the image, which makes it possible to improve the accuracy when the MRI image is compared with the standard brain image.

The tissue segmentation function 212 generates a gray matter brain image and a white matter brain image obtained by extracting a gray matter (GM) and a white matter (WM) from the MRI image subjected to the image reconstruction. The T1-weighted image includes three types of tissues of a white matter that has a high signal value corresponding to a nerve fiber, a gray matter that has an intermediate signal value corresponding to a nerve cell, and cerebrospinal fluid (CSF) that has a low signal value. Therefore, a process which extracts each of the gray matter, the white matter, and the cerebrospinal fluid is performed with a focus on a difference between the signal values.

The anatomical standardization function 213 performs anatomical standardization on the extracted gray matter brain image, white matter brain image, and cerebrospinal fluid image. The anatomical standardization is aligning voxels with those in the standard brain image. In this embodiment, anatomical standardization using diffeomorphic anatomical registration through exponentiated lie algebra (DARTEL) is performed. DARTEL is an algorithm for performing nonlinear transformation using a large number of parameters.

The smoothing function 214 performs an image smoothing process on the gray matter brain image and the white matter brain image subjected to the anatomical standardization using DARTEL to improve an S/N ratio. Individual differences that are not completely matched with each other by the anatomical standardization process can be reduced by performing the image smoothing in this way.

The density value correction function 215 performs density value correction to correct voxel values of the entire brain to be matched with a distribution of voxel values in an image group of healthy persons. The gray matter brain image, the white matter brain image, and the cerebrospinal fluid image subjected to the density value correction are output to the image feature amount calculation function 22.

FIG. 3 is a diagram illustrating an example of a configuration of the image feature amount calculation function 22 according to a first embodiment. The image feature amount calculation function 22 includes an atrophy score calculation function 221, an ROI specification function 222, and an atrophy degree calculation function 223. The image feature amount calculation function 22 calculates the degree of atrophy (degree of ROI atrophy) calculated by the atrophy degree calculation function 223 as an image feature amount.

The atrophy score calculation function 221 compares the MRI image of the subject with the MRI images of the healthy persons with reference to the MRI images of the healthy persons recorded on the control group database 32 to calculate an “atrophy score” indicating the degree of brain atrophy of the subject. In this embodiment, a “Z score”, which is a statistical index, is used as the atrophy score. Specifically, the statistical comparison between the gray matter brain image and the white matter brain image of the subject subjected to the anatomical standardization, the image smoothing, and the like by the image processing function 21 and an MRI image group of the gray matter and white matter of a large number of healthy persons recorded on the control database 32 is performed to calculate the Z scores of the gray matter and the white matter for all voxels of the MRI image or voxels in a specific region.

The Z score can be calculated by the following expression:

Z score=(μ(x,y,z)−P(x,y,z))/σ(x,y,z).

μ indicates the average of the voxel values of the MRI image group of the healthy persons, σ indicates a standard deviation of the voxel values of the MRI image group of the healthy persons, and P indicates the voxel value of the MRI image of the subject. (x, y, z) is coordinate values of the voxel. The Z score is a value obtained by scaling the difference between the voxel value of the image of the subject and the average of the corresponding voxel values of the image group of the healthy persons with the standard deviation and indicates the degree of relative decrease in the volume of the gray matter and the white matter. The use of the Z score makes it possible to compare the MRI image of the subject with an image group of the healthy persons to quantitatively analyze what changes occur in which parts. For example, a voxel with a positive Z score indicates a region with atrophy as compared to the standard brains of a healthy person group, and it can be interpreted that, as the value is larger, the amount of deviation is statistically larger. For example, when the Z score is “2”, the value is more than twice the standard deviation from the average, and it is estimated that there is a statistically significant difference with a risk rate of approximately 5%. In addition, the atrophy score is not limited to the Z score. An index that can be used to determine the magnitude of the voxel values in the image of the subject and the image of the healthy person may be used as the atrophy score indicating the degree of atrophy (for example, a t-score and the like).

The ROI specification function 222 specifies a brain part (region of interest: ROI) specific to each disease. For example, the ROI specification function 222 can specify the region of interest related to each disease on the basis of statistical processing. Specifically, in a case where the region of interest corresponding to a certain disease is specified, a two-sample t-test that statistically tests a significant difference between two groups on a voxel-by-voxel basis is performed on an MRI image group (disease image group) of a subject with the disease and an MRI image group (non-diseased subject image group) of a subject (for example, a healthy person) without the disease, and the voxels, which have been found to have a significant difference therebetween, are considered as characteristic voxels for the disease. Then, a set of the coordinates of the voxels is specified as the region of interest corresponding to the disease. A plurality of regions of interest may be provided. In addition, the region of interest may be specified in consideration of both a significance level and an empirical rule. Further, the region of interest may be specified, for example, only from a disease image (or a disease image group). For example, for the disease image (or the disease image group), a part that has large atrophy in correlation to the size of atrophy in the entire brain may be specified as the region of interest.

The ROI specification function 222 may read and use the region of interest recorded on the ROI 31 for image feature amount calculation. In addition, the ROI specification function 222 may specify the region of interest on the basis of information (atlas) that spatially partitions the brain with reference to the brain atlas database 35. For example, an automated anatomical labeling (AAL) atlas, a Brodmann atlas, a LONI probabilistic brain atlas (LPBA40), a Talairach atlas, or the like can be used as the brain atlas.

The atrophy degree calculation function 223 calculates the degree of atrophy of each region of interest specified by the ROI specification function 222. The degree of atrophy can be, for example, the average value of positive Z scores in the region of interest. For example, when the region of interest is the hippocampus, the average value of the positive Z scores in the hippocampus can be calculated as the degree of atrophy of the hippocampus. In addition, the degree of atrophy is not limited to the “average value of the positive Z scores” in the region of interest. For example, a predetermined threshold value for the Z score may be determined, and the degree of atrophy may be the average value of Z scores exceeding the threshold value. Alternatively, the degree of atrophy may be the average value of the Z scores or may be the maximum value of the Z scores. Further, the proportion of voxels having Z scores exceeding the threshold value to the total number of voxels in the region of interest may be used. Furthermore, the sum or average of the voxel values in the region of interest may simply be used as the image feature amount without a comparison to the control group. The degree of atrophy (image feature amount) of the brain part calculated by the atrophy degree calculation function 223 is output to the prediction function 23.

FIG. 4 is a diagram illustrating an example of a configuration of the prediction function 23. The prediction function 23 includes a scaling function 231 and a trained prediction model 232. The scaling function 231 has the function of an acquisition unit that acquires subject data related to the brain of the subject. The subject data includes an image feature amount and subject information. Hereinafter, the subject data will be described.

FIG. 5 is a diagram illustrating an example of a configuration of the subject data. The subject data is a set of data having different units and attributes and can be broadly classified into three categories of an image feature amount, an image capture parameter, and subject information. The image feature amount can be further classified into the degree of atrophy of a brain part and a feature vector described below which are calculated by the image feature amount calculation function 22. The feature vector will be described in detail below.

The subject data includes not only subject data at one time point but also subject data at a plurality of time points. For example, as a first example, subject data at two time points, that is, subject data at the current time and subject data six months ago can be input to the prediction function 23, and prediction can be performed. As a second example, subject data at three time points, that is, at the current time, six months ago, and one year ago can be input to the prediction function 23, and prediction can be performed. As a third example, subject data at the current time, subject data at a time point that was a period Δt before the current time, and the period Δt can be input to the prediction function 23, and prediction can be performed.

The image capture parameter includes, for example, model information of the MRI apparatus and an imaging protocol (for example, the strength of a magnetic field, a sequence type, imaging parameters, and the like).

The subject information can be further classified into neuropsychological test information, clinical information, and biochemical test information.

The neuropsychological test information includes, for example, Alzheimer's Disease Assessment Scale-cognitive subscale (ADAS-cog), Mini-Mental State Examination (MMSE), Clinical Dementia Rating (CDR), Functional Activity Questionnaire (FAQ), Geriatric Depression Scale (GDS), a neuropsychological battery (for example, a battery obtained by combining several tests such as Logical Memory IA Immediate Recall, Logical Memory IIA Delayed Recall, WAIS-III, Clock Drawing/Clock Copying, Verbal Fluency Task, Trail Making Test A&B, and Boston Naming Test), and the like. MMSE can examine the severity of dementia. A subject who has scored 24 points or less out of 30 points is determined to be suspected of having dementia, and a subject who has scored 0 to 10 points is determined to have severe dementia. In CDR, a subject who has scored 0 points is determined to be a healthy person, a subject who has scored 0.5 points is determined to be suspected of having dementia, a subject who has scored 1 point is determined to have mild dementia, a subject who has scored 2 points is determined to have moderate dementia, and a subject who has scored 3 points is determined to have advanced dementia. GDS is evaluated on a scale of 0 to 15 points. In a case where the score is 6 or more, “depression” is suspected. ADAS-cog is evaluated on a scale of 0 to 70 points. As the score is higher, the degree of dementia is more severe. In the neuropsychological battery, the tests to be combined can be changed as appropriate depending on the status of the subject's dementia.

The clinical information includes, for example, the age, gender, height, weight, BMI, years of education, medical history (presence or absence of diabetes and the like), family history, presence or absence of dementia in the family, vital signs (blood pressure, pulse, body temperature, and the like) of the subject.

The biochemical test information includes blood test results, cerebrospinal fluid test results (CSF-TAU (including T-TAU and P-TAU) and CSF-Aβ), ApoE genotype, and the like.

The scaling function 231 can acquire at least one of the neuropsychological test information, the clinical information, and the biochemical test information. For example, the scaling function 231 may acquire only the clinical information or may acquire the neuropsychological test information or the biochemical test information. In addition, each of the neuropsychological test information, the clinical information, and the biochemical test information to be acquired may include only some of the above-described items.

The scaling function 231 performs scaling to uniformize the image feature amounts (also including the image capture parameter in this case) and the subject information because they have different units and attributes. Examples of the scaling method include standardization, normalization, and the like. The standardization is a scaling method in which the mean is 0 and the variance is 1. A value x′ after scaling can be calculated by an expression of x′=(x−μ)/σ. Here, x indicates a value before scaling, μ indicates the mean, and σ indicates the standard deviation.

The normalization is a scaling method in which the minimum value is 0 and the maximum value is 1. A value x′ after scaling can be calculated by an expression of x′=(x−xₘᵢₙ)/(xₘₐₓ−xₘᵢₙ). Here, x indicates a value before scaling, xₘₐₓ indicates the maximum value of x, and xₘᵢₙ indicates the minimum value of x. The scaling function 231 outputs subject data X=(x1, x2, . . . , xn) after the scaling to the trained prediction model 232.

The trained prediction model 232 has the function of a prediction unit and predicts the brain disease of the subject on the basis of the subject data. A prediction model of the trained prediction model 232 is represented by f(X, θ). When subject data X=(x1, x2, . . . , xn) of one subject is input to the trained prediction model 232, the trained prediction model 232 outputs a prediction result y and a probability estimation value p. The prediction result y is the presence or absence of the AD conversion of the subject after a certain number of years (prediction period: T years). The trained prediction model 232 performs binary classification identification to predict the presence or absence of AD conversion. For example, in a case where y is 1, AD conversion can be predicted to be present after T years (within T years). In a case where y is 0, AD conversion can be predicted to be absent after T years (within T years). For example, the probability estimation value p indicates the probability that AD conversion will be present in a case where y is 1 and indicates the probability that AD conversion will be absent in a case where y is 0. The probability estimation value p is in the range of 0 to 1. In addition, the trained prediction model 232 may be provided for each prediction period T. Alternatively, one trained prediction model 232 may predict the presence or absence of AD conversion in a plurality of prediction periods T.

Further, in the example illustrated in FIG. 4, both the image feature amount and the subject information are used as the subject data. However, the present invention is not limited thereto, and only the image feature amount may be used as the subject data. In this case, the subject information is not essential.

The trained prediction model 232 may be any prediction model that performs the binary classification identification. For example, machine learning models, such as random forest, support vector machine (SVM), Adaboost, gradient boosting, logistic regression, a decision tree, a neural network, deep learning, and a plurality of ensembles of these models, can be used.

Next, a method for generating the trained prediction model 232 will be described. A learning data set is composed of a large number of pieces of case data. It is assumed that the scaled subject data of the subject is X′=(x′1, x′2, . . . , x′n), a follow-up is performed on the subject, and the presence or absence of AD conversion after T years is y′. Here, it is assumed that y′=1 indicates the presence of AD conversion and y′=0 indicates the absence of AD conversion.

The learning processing function 26 estimates a model parameter θ at which y′=f(X′, θ) is established, using the subject data X′ of a large number of subjects and the presence or absence y′ of the AD conversion of the subject after T years as the learning data set, X′ as an explanatory variable, and y′ as an objective variable. This makes it possible to generate the trained prediction model 232. The generated trained prediction model 232 can be stored in the trained model parameter 33.

The prediction basis calculation function 24 has the function of a contribution calculation unit and calculates the degree of contribution of the subject data, which contributes to the prediction result of the trained prediction model 232. For example, it is assumed that, in a case where the subject data X=(x1, x2, x3, . . . , xn) is input to the trained prediction model 232, the prediction result is y. The prediction basis calculation function 24 calculates the degree of contribution C=(c1, c2, c3, . . . , cn) having the subject data X=(x1, x2, x3, . . . , xn) as the basis for prediction for the prediction result y. For example, the degree of contribution c1 is the degree of contribution having the subject data x1 as the basis for prediction.

The degree of contribution can be calculated by, for example, a shapley additive explanations (SHAP) technique, a local interpretable model-agnostic explanations (LIME) technique, an Explainable Boosting Machine technique, and the like. The SHAP technique is a method that proportionally divides an increase or decrease in the prediction probability of the data to be explained from the average by the influence of each variable in the data. The LIME technique is a method that locally approximates a trained model by a simple model (a model that can be applied only to the data to be explained and its surroundings) and calculates a coefficient of the approximate model as the degree of contribution. The Explainable Boosting Machine technique is a method that combines a generated additive model (GAM) and a gradient boosting method to achieve both the accuracy of gradient boosting and the interpretability of GAM. The SHAP technique will be described below.

Hereinafter, for convenience, three pieces of data x1, x2, and x3 are considered as the subject data. In the SHAP technique, the concept of marginal contribution is introduced. For example, in the case of the subject data x1, the marginal contribution indicates how much the prediction result y increases when the subject data x1 is input to the trained prediction model 232. Furthermore, in a case where the subject data x1 is input to the trained prediction model 232, the prediction result y changes depending on which of the subject data x2 and the subject data x3 has already been input. In a case where there are three pieces of subject data, there are six input orders (addition orders) of the subject data x1, the subject data x2, and the subject data x3. The degree of contribution c1 of the subject data x1 can be calculated as the average value of marginal contributions calculated for all of the orders.

FIG. 6 is a diagram illustrating an example of the calculation of the degree of contribution. The subject data x1, the subject data x2, and the subject data x3 are added in the following six orders: x1→x2→x3; x1→x3→x2; x2→x1→x3; x2→x3→x1; x3→x1→x2; and x3→x2→x1. When the marginal contribution of x1 in each addition order is ϕ11 to ϕ16, the degree of contribution c1 of x1 can be calculated as the average value of ϕ11 to ϕ16. In addition, when the marginal contribution of x2 in each addition order is ϕ21 to ϕ26, the degree of contribution c2 of x2 can be calculated as the average value of ϕ21 to ϕ26. When the marginal contribution of x3 in each addition order is ϕ31 to ϕ36, the degree of contribution c3 of x3 can be calculated as the average value of ϕ31 to ϕ36.

The prior knowledge collation function 25 collates the basis for prediction with the prior knowledge database 34.

FIG. 7 is a diagram illustrating an example of a configuration of the prior knowledge database 34. In the prior knowledge database 34 illustrated in FIG. 7, a prediction task corresponds to AD conversion. The same prior knowledge database 34 can also be constructed for other prediction tasks. As illustrated in FIG. 7, in the prior knowledge database 34, prior knowledge (explanatory text) is registered for each data item, and the strength of the prior knowledge (reliability as evidence) is associated with each data item. The strength of the prior knowledge can be classified into, for example, “high”, “medium”, and “low”. However, the strength is not limited thereto and may be represented by a numerical value and the like. Further, a URL of a reference destination of a detailed explanation related to the prior knowledge is also associated with each data item. Items related to AD conversion are given as examples of the data item. Examples of the data item include the degree of hippocampal atrophy, the degree of whole brain atrophy, the degree of medial temporal lobe atrophy, amyloid PET, MMSE, CDR, logical memory, GDS, and the number of years of education. In addition, the data items are not limited to the examples illustrated in FIG. 7.

As illustrated in FIG. 7, the data item “degree of hippocampal atrophy” is associated with an explanatory text “There is strong evidence that hippocampal atrophy increases risk” as the prior knowledge. In this case, the strength of the knowledge is “high”. In addition, the data item “MMSE” is associated with an explanatory text “There is strong evidence that a decrease in MMSE increases risk” as the prior knowledge. In this case, the strength of the knowledge is “high”. Further, the data item “logical memory” is associated with an explanatory text “There is evidence that logical memory increases risk” as the prior knowledge. In this case, the strength of the knowledge is “medium”.

The prior knowledge collation function 25 has the function of a specification unit and specifies a data item corresponding to subject data, which contributes to the prediction result of the trained prediction model 232, in the subject data with reference to the prior knowledge database 34. When the subject data is X=(x1, x2, x3, . . . , xn), an item of each piece of data x1, x2, x3, . . . , xn is the data item, and the value of each piece of data is the value of the data item. For example, when the item of the data x1 is the “degree of hippocampal atrophy”, the value of the data item is the value of the degree of hippocampal atrophy.

More specifically, the prior knowledge collation function 25 can specify a data item corresponding to the subject data on the basis of the degree of contribution calculated by the prior knowledge collation function 25 and a predetermined contribution degree threshold value and read prior knowledge corresponding to the specified data item from the prior knowledge database 34.

FIG. 8 is a diagram illustrating an example of a method for reading the prior knowledge. It is assumed that the subject data (data item) is x1, x2, . . . , xi, . . . , xn and the degrees of contribution of each piece of subject data are c1, c2, . . . , ci, . . . , cn.

It is assumed that threshold values for each degree of contribution are Th1, Th2, Thi, . . . , Thn. In addition, the threshold values Th1, Th2, . . . , Thi, . . . , Thn for each degree of contribution may be the same value. As illustrated in FIG. 8, when C1>Th1 is satisfied, prior knowledge corresponding to the subject data x1 is read from the prior knowledge database 34. Further, when C2<Th2 is satisfied, prior knowledge corresponding to the subject data x2 is not read. The reading method is performed in the other cases in the same manner as described above. In this way, the prior knowledge collation function 25 can select prior knowledge corresponding to subject data, whose degree of contribution is greater than the threshold value, from the subject data x1, x2, . . . , xi, . . . , xn.

With the above-described configuration, the relationship (association) between the basis for prediction and conventionally known prior knowledge is shown for the prediction result by the prediction function 23 such that a user, such as a doctor, can know how much known evidence supports the prediction result. For example, when the degree of match of the basis for prediction (reason for prediction) with the known evidence is high, it can be determined that the reliability of the basis for prediction is high.

The prior knowledge collation function 25 has the function of an output unit and can output the specified data item and the prior knowledge related to the brain disease to the user interface unit 10 in association with each other. The prior knowledge collation function 25 can output the subject data corresponding to the specified data item. Further, the prior knowledge collation function 25 may output the degree of match (degree of association) between the specified data item and the prior knowledge. The degree of match indicates how much the basis for prediction (reason for prediction) is matched with the prior knowledge.

The prior knowledge collation function 25 has the function of an association degree calculation unit and can calculate the degree of match (degree of association) on the basis of at least one of the degree of contribution of the subject data corresponding to the data item and the strength (reliability) of the prior knowledge as evidence. For example, when the degree of contribution is C and the strength of prior knowledge is S, the degree of match E can be calculated by an expression of E=α·C+β·S. α and β are weighting coefficients and may be 0. However, α+β≠0 is established. The degree of match may be classified into, for example, three categories of “high”, “medium”, and “low” depending on the value of E.

The prediction result display function 13 can display the prediction result in which the specified data item is associated with the prior knowledge related to the brain disease. In addition, the prediction result can include information output by the prior knowledge collation function 25.

FIG. 9 is a diagram illustrating a first example of the display of the prediction result. An “Alzheimer's type dementia conversion prediction result” is a result of analyzing and predicting subject data of a certain subject (patient) by prediction function 23 and indicates the probability of converting to Alzheimer's type dementia after a certain number of years. In the example illustrated in FIG. 9, the conversion rates after 2 years and after 5 years are displayed. However, either one of the conversion rates may be displayed. In addition, the prediction period may be one year, three years, and the like. The conversion rate indicates the probability estimation value p output by the trained prediction model 232.

A “reason for prediction” indicates the basis of the subject data on which the prediction function 23 reached the prediction. An “item” is the data item of the subject data, a “measured value” is the value of the data item, and a “contribution to prediction” is the degree of contribution and indicates how much each item of the subject data contributes to prediction. In “known evidence”, “explanation” is displayed as the prior knowledge. The “explanation” is associated with the “reason for prediction”. A “degree of match” indicates how much the “reason for prediction” is matched with the prior knowledge. The “degree of match” can be expressed as, for example, “high”, “medium”, or “low”, but is not limited thereto. The “degree of match” may be expressed by a numerical value. In “details”, an arrow icon for displaying a detailed explanation is provided. The icon can be operated to display more detailed information related to the explanation.

The prediction result display function 13 has the function of a display unit and can display the data item, the measured value (the value of the subject data), and the prior knowledge corresponding to the data item in association with one another in the order of the contribution to prediction corresponding to the data item (the degree of contribution of the subject data to the prediction result). In the example illustrated in FIG. 9, each piece of information is displayed in the order of the degrees of contribution of 5, 4, 3.6, 2.2, 2, . . . . Therefore, the user, such as a doctor, can visually recognize the reason for prediction in the order of the contribution to the prediction result and can easily see an important reason for prediction and known evidence. In addition, in the example illustrated in FIG. 9, it can be seen that the magnitude of the measured value of the “degree of hippocampal atrophy” is the largest influencing factor that acts on the positive side of the prediction result and that, since the measured value of the “degree of temporal lobe atrophy” is small, the magnitude of the measured value is a factor that acts on the negative side (direction that does not contribute to AD conversion) of the prediction result.

The display of the prediction result illustrated in FIG. 9 enables the doctor to know how much known evidence supports the prediction result of the diagnosis support device 50. It can be seen that, when the degree of match of the basis for prediction (reason for prediction) of the diagnosis support device 50 with known evidence is high, the reliability of the basis for prediction is high.

In addition, when the degree of match with known evidence is low, it can be interpreted that the diagnosis support device 50 shows a new evidence hypothesis, but this is likely to be a result of bias in learning data. Therefore, when the basis for prediction is contrary to common sense or logic, additional and more detailed tests may be performed in order to further improve the reliability, without making the determination on the basis of only the prediction result.

As described above, according to this embodiment, since the basis for predicting a brain disease is associated with prior knowledge, the doctor can make a diagnosis with reference to the basis for prediction, and it is possible to support the doctor's diagnosis.

### Second Embodiment

In the first embodiment, the degree of ROI atrophy is used as the image feature amount. However, the image feature amount is not limited to the degree of ROI atrophy. In a second embodiment, a configuration in which a feature vector is used as the image feature amount will be described.

FIG. 10 is a diagram illustrating an example of a configuration of an image feature amount calculation function 22 according to the second embodiment. In the second embodiment, the image feature amount calculation function 22 can be configured by a convolutional neural network (CNN) and includes an input layer 22a, a plurality of (for example, 17 or the like) convolutional layers 22b, a pooling layer 22c, fully connected layers 22d and 22e, and an output layer 22f. In addition, the configuration of the CNN is illustrative and is not limited to the example illustrated in FIG. 10. For example, VGG16, ResNet, DenseNet, EfficientNet, AttResNet, and the like may be used. A standardized gray matter image is input from the image processing function 21 to the input layer 22a. That is, an image obtained by anatomically standardizing the gray matter image among the gray matter (GM), the white matter (WM), and the cerebrospinal fluid (CSF) which are tissue segmentation results is input to the input layer 22a. The output layer 22f outputs the presence or absence of AD conversion which is the prediction result. The prediction result of the output layer 22f is used in a prediction basis calculation process which will be described below.

The image feature amount calculation function 22 calculates, as the image feature amount, a feature vector that has, as an element, the value of each node of the fully connected layer 22e immediately before the output layer 22f. When the number of nodes in the fully connected layer 22e is N, it is possible to obtain N image feature amounts. The image feature amount calculation function 22 outputs the calculated feature vector to the prediction function 23.

The prediction function 23 acquires the feature vector output by the image feature amount calculation function 22 as the image feature amount and performs the same process as that in the first embodiment.

In order to generate a trained CNN model, parameters of the CNN may be updated using a learning data set such that a prediction error (for example, a cross-entropy error) of the prediction result (the presence or absence of AD conversion) by the CNN is minimized. The learning processing function 26 can generate the trained CNN model.

FIG. 11 is a diagram illustrating an example of a configuration of a prediction basis calculation function 24 according to the second embodiment. The prediction basis calculation function 24 includes a gradient calculation function 241, a weighting calculation function 242, an adder 243, and an ReLU 244. It is assumed that feature maps output from the convolutional layer 22b of the image feature amount calculation function 22 (CNN) are A1, A2, A3, . . . , Ak. k indicates the number of channels. A k-channel feature map is generated by using k filters in a convolution operation. For example, the feature map output from a layer, which is the final layer of the convolutional layer and is on the input side of the fully connected layer, can be used. The reason is that position information of the image is lost in the fully connected layer and the features of the image can be well abstracted in the final layer of the convolutional layer. In addition, the layer is not limited to the final layer and may be any convolutional layer.

How much the feature maps A1, A2, A3, . . . , Ak influence the prediction result from the output layer 22f is calculated using a gradient. The gradient calculation function 241 calculates gradients α1, α2, α3, . . . , αk of the feature maps A1, A2, A3, . . . , Ak, respectively. The gradient operation is an operation that calculates how much the prediction result changes when each element of the feature map changes minutely and performs smoothing in the feature map.

Then, the importance of the feature map is calculated using the result of the gradient operation. Specifically, the weighting calculation function 242 performs a weighting operation by multiplying each of the feature maps A1, A2, A3, . . . , Ak by each global average pooling value (M1, M2, M3, . . . , Mk which are the average values of each gradient in the image) of the gradients α1, α2, α3, . . . , αk. A heat map is generated by adding the weighted feature maps α1·A1, α2·A2, α3·A3, . . . , αk·Ak with the adder 243 and passing the result through the ReLU (activation function) 244. The heat map is obtained by imaging and visualizing which portion of the image is used as the basis for determination with a focus on the feature map extracted by the convolutional layer 22b as the basis for prediction. The basis for prediction can be visualized by scaling the image size of the heat map to the size of the input standardized gray matter image and superimposing the heat map on the standardized gray matter image.

The heat map has three-dimensional heat map information in which the basis (degree of influence) for the prediction result is represented by a numerical value (also referred to as a heat map value), corresponding to each coordinate position (x, y). A feature portion indicated by the heat map information can indicate the height of the feature according to the magnitude of the heat map value. A display aspect (for example, a color, density, or the like) can be changed depending on the importance of the basis for determination. In addition to Grad-CAM described in FIG. 11, Guided Grad-CAM, a Guided Backprop technique, or the like may be used as the visualization method. The Guided Backprop technique is a kind of gradient-based highlighting method in which, as the amount of change when the value of certain data is minutely changed, the degree of contribution is considered to be higher.

FIG. 12 is a diagram illustrating a second example of the display of the prediction result. The second example differs from the first example illustrated in

FIG. 9 in that an image region (an image region indicating the basis for prediction) and the name of a brain atlas corresponding to the image region are displayed. Each image in the image region is obtained by overlaying and displaying, on a standard brain image, regions (regions 1 to 3) divided by a process of clustering heat map values with a predetermined threshold value. In addition, the name of an atlas at the corresponding coordinates is displayed on the basis of the coordinates of the regions 1 to 3. In the example illustrated in FIG. 12, the regions 1 to 3 are “hippocampus, parahippocampal gyrus”, “hippocampus, lingual gyrus”, and “precuneus, calcarine sulcus”, respectively.

As described above, the doctor can not only see which specific region of the brain has a strong influence on the prediction on the image, but also check whether evidence linked to the atlas is present or absent by associating the coordinates with the atlas.

FIG. 13 is a diagram illustrating an example of a configuration of a diagnosis support device 80 according to this embodiment. For example, a personal computer or the like can be used as the diagnosis support device 80. The diagnosis support device 80 can include, for example, the processing unit 20 and can be composed of a CPU 81, a ROM 82, a RAM 83, a GPU 84, a video memory 85, a recording medium reading unit 86, and the like. The recording medium reading unit 86 (for example, an optical disk drive) can read a computer program (program product) recorded on a recording medium 90 (for example, an optically readable disk storage medium such as a CD-ROM) and store the computer program in the RAM 83. Here, the computer program includes processing procedures illustrated in FIGS. 15 and 16 which will be described below. The computer program may be stored in a hard disk (not illustrated). When the computer program is executed, it may be stored in the RAM 83.

The CPU 81 can execute the computer program stored in the RAM 83 to perform each process of the image processing function 21, the image feature amount calculation function 22, the prediction function 23, the prediction basis calculation function 24, the prior knowledge collation function 25, and the learning processing function 26. The video memory 85 can temporarily store data for various types of image processing and processing results. In addition, instead of the configuration in which the computer program is read by the recording medium reading unit 86, the computer program can also be downloaded from another computer, a network device, or the like via a network such as the Internet.

In the above-described example, the diagnosis support device 50 includes the user interface unit 10, the processing unit 20, and the database unit 30. However, the present invention is not limited thereto. For example, the user interface unit 10, the processing unit 20, and the database unit 30 can be distributed as follows.

FIG. 14 is a diagram illustrating an example of a configuration of a diagnosis support system. The diagnosis support system includes a terminal device 100, a diagnosis support server 200, and a data server 300. The terminal device 100, the diagnosis support server 200, and the data server 300 are connected via a communication network 1 such as the Internet. The terminal device 100 corresponds to the user interface unit 10 and is configured by a personal computer or the like. The diagnosis support server 200 corresponds to the processing unit 20, and the data server 300 corresponds to the database unit 30. Since the functions of the terminal device 100, the diagnosis support server 200, and the data server 300 are the same as those of the user interface unit 10, the processing unit 20, and the database unit 30, a description thereof will not be repeated.

Next, a process of the diagnosis support device 50 will be described.

FIG. 15 is a diagram illustrating a procedure of a prediction process. The processing unit 20 acquires a medical image of the subject (S11) and acquires subject information of the subject (S12). The processing unit 20 performs image reconstruction on the acquired medical image (S13) and performs tissue segmentation (S14). The tissue segmentation is, for example, a process of separating and extracting gray matter, white matter, and cerebrospinal fluid.

The processing unit 20 performs anatomical standardization for each segmented tissue (S15) and calculates an image feature amount from the medical image subjected to the anatomical standardization (S16). The image feature amount may be, for example, the degree of ROI atrophy or a feature vector.

The processing unit 20 scales the image feature amount and the subject information (S17), inputs the scaled subject data to the prediction function 23, and performs a prediction result calculation process (S18). The processing unit 20 performs a prediction basis calculation process (S19). The prediction basis calculation process includes a process of calculating the degree of contribution of each piece of subject data to the prediction result.

The processing unit 20 performs collation with prior knowledge on the basis of the calculated degree of contribution (S20). In the collation with prior knowledge, The processing unit 20 specifies a data item corresponding to the subject data on the basis of the calculated degree of contribution and a predetermined contribution degree threshold value and reads prior knowledge corresponding to the specified data item from the prior knowledge database 34.

The processing unit 20 outputs the prediction result (S21) and ends the process. The prediction result is as illustrated in FIG. 9 or FIG. 12.

As described above, the computer program causes the computer to execute a process that acquires subject data related to the brain of the subject, predicts a brain disease of the subject on the basis of the subject data, specifies a data item corresponding to subject data, which contributes to the prediction result of the brain disease, in the subject data, and outputs the specified data item and the prior knowledge related to the brain diseases in association with each other.

FIG. 16 is a diagram illustrating a procedure of a process of generating the trained prediction model 232. The processing unit 20 acquires a medical image of the subject from a large number of pieces of case data (S31) and acquires subject information of the subject (S32). The processing unit 20 acquires training data collected through the follow-up of the subject (S33). The training data is, for example, data indicating whether or not the subject has converted to AD.

The processing unit 20 performs image reconstruction on the acquired medical image (S34) and performs tissue segmentation (S35). The tissue segmentation is, for example, a process of separating and extracting gray matter, white matter, and cerebrospinal fluid. The processing unit 20 performs anatomical standardization for each segmented tissue (S36) and calculates an image feature amount from the medical image subjected to the anatomical standardization (S37). The image feature amount may be, for example, the degree of ROI atrophy or a feature vector.

The processing unit 20 determines whether or not there is other learning data (S38). In a case where there is learning data (YES in S38), the processing unit 20 repeats the processes after step S31. In a case where there is no learning data (NO in S38), the processing unit 20 scales the image feature amount and the subject information (S39). In a case where subject data for learning is input to a learning model, the processing unit 20 updates the internal parameters of the learning model such that data output by the learning model is close to the training data (S40).

The processing unit 20 determines whether or not the value of a loss function indicating an error between the data output by the learning model and the training data is within an allowable range (S41). In a case where the value of the loss function is not within the allowable range (NO in S41), the processing unit 20 repeats the processes after step S40. In a case where the value of the loss function is within the allowable range (YES in S41), the processing unit 20 stores the generated trained prediction model 232 in the trained model parameter 33 (S42) and ends the process.

In the above-described embodiment, AD conversion prediction has been described as the prediction task. However, this embodiment can also be applied to other prediction tasks. Other prediction tasks will be described below.

FIG. 17 is a diagram illustrating other prediction tasks. Other prediction tasks include, for example, AD conversion period prediction, amyloid ß deposition prediction, AD/DLB disease differentiation, AD severity prediction, tau abnormality prediction, brain age prediction, and the like. Hereinafter, the outline, evaluation method, objective variables, and the like of each prediction task will be described.

The AD conversion period prediction predicts the period until conversion to AD in the future as a numerical value. Knowing the time when the subject is likely to convert to AD can be useful for reviewing future lifestyle habits and formulating long-term treatment plans. The evaluation is regression, and the objective variable is the length of the period until AD conversion.

The amyloid β deposition prediction predicts amyloid β deposition from test results (for example, an MRI image, subject information, and the like) other than amyloid β (Aβ). For example, the distribution state of amyloid β in the brain of the subject can be estimated to estimate symptoms of diseases related to amyloid β. The diseases related to amyloid β include, for example, neurodegenerative diseases such as mild cognitive impairment (MCI), mild cognitive impairment due to Alzheimer's disease, prodromal Alzheimer's disease, a pre-symptomatic stage of Alzheimer's disease/preclinical AD, Parkinson's disease, multiple sclerosis, cognitive decline, cognitive dysfunction, and diseases related to amyloid positive/negative. The evaluation method is binary classification, and the objective variable is amyloid ß positive or negative. Training data during learning can be determined using a threshold value from the results of both amyloid PET (imaging test) and a cerebrospinal fluid test. Prediction result 1 can be amyloid β positive or negative, and prediction result 2 can be positive probability.

The AD/DLB disease differentiation predicts whether the disease is Alzheimer's disease (AD) or dementia with Lewy bodies (DLB). DLB has some similarities to AD in the trend of brain atrophy, but a treatment method for DLB is different from that for AD. Therefore, it is important to perform differentiation. The evaluation method is binary classification, and the objective variable is AD or DLB. Prediction result 1 can be AD or DLB, and prediction result 2 can be AD probability and DLB probability.

The AD severity prediction predicts the severity (mild, moderate, and severe) of Alzheimer's disease only from brain images. The difference between the brain image and clinical progress can be investigated and used for treatment. The evaluation method is regression for numerical prediction. The objective variable is clinical dementia rating (CDR). A CDR of 0 indicates good health, a CDR of 0.5 indicates suspected dementia, a CDR of 1 indicates mild dementia, a CDR of 2 indicates moderate dementia, and a CDR of 3 indicates advanced dementia. The explanatory variable is the image feature amount of the brain image.

The tau abnormality prediction predicts abnormalities in tau deposition from information other than tau PET. The tau is a protein that is expressed in, for example, nerve cells of the central nervous system or the peripheral nervous system, and tau abnormalities are considered to be a cause of a neurodegenerative disease such as Alzheimer's disease. The evaluation method is regression, and the objective variable is a tau PET standardized uptake value ratio (SUVR). For example, the SUVR can be calculated by dividing the sum of the SUVs (degree of accumulation of tau) of four parts of the cerebral gray matter (prefrontal cortex, anterior and posterior cingulate cortex, parietal lobe, and lateral temporal lobe) by the SUV of a specific reference region (for example, cerebellum or the like).

The brain age prediction predicts “brain age” from brain images. Even in the case of a healthy person, knowing the state (brain age) of the person's brain can be useful for checking the person's health such as reviewing lifestyle habits. The evaluation method is regression, and the objective variable is age. Training data during learning can be the actual age at the time when the brain image was captured. The explanatory variable is the image feature amount of the brain image.

According to this embodiment, it is possible to visualize and present the importance of the factors that are the basis for the prediction result of the prediction task. Therefore, the doctor can diagnose brain diseases and the like with reference to the basis for prediction, and it is possible to support the doctor's diagnosis.

A diagnosis support device according to this embodiment includes: an acquisition unit acquiring subject data related to a brain of a subject; a prediction unit predicting a brain disease of the subject on the basis of the subject data; a specification unit specifying a data item corresponding to subject data, which contributes to a prediction result of the prediction unit, in the subject data; and an output unit outputting the data item specified by the specification unit and prior knowledge related to the brain disease in association with each other.

In the diagnosis support device according to this embodiment, the acquisition unit acquires, as the subject data, an image feature amount calculated on the basis of a medical image related to the brain of the subject.

In the diagnosis support device according to this embodiment, the image feature amount includes a degree of atrophy of a part of the brain.

In the diagnosis support device according to this embodiment, the acquisition unit acquires, as the subject data, subject information including at least one of test information and clinical information related to the brain of the subject.

The diagnosis support device according to this embodiment further includes: a storage unit storing the prior knowledge as known evidence related to the brain disease in association with a data item related to the brain; and a contribution degree calculation unit calculating a degree of contribution of the subject data, which contributes to the prediction result. The specification unit specifies the data item corresponding to the subject data on the basis of the degree of contribution and a predetermined contribution degree threshold value, and the output unit reads prior knowledge corresponding to the specified data item from the storage unit and outputs the prior knowledge.

In the diagnosis support device according to this embodiment, the output unit outputs a degree of association between the data item and the prior knowledge.

The diagnosis support device according to this embodiment further includes an association degree calculation unit calculating the degree of association on the basis of at least one of the degree of contribution of the subject data corresponding to the data item and a reliability of the prior knowledge as evidence.

In the diagnosis support device according to this embodiment, the output unit outputs the subject data corresponding to the data item.

The diagnosis support device according to this embodiment further includes a display unit displaying the data item, the subject data, and the prior knowledge corresponding to the data item in association with one another in an order of the degree of contribution of the subject data corresponding to the data item to the prediction result.

A computer program according to this embodiment causes a computer to execute a process including: acquiring subject data related to a brain of a subject; predicting a brain disease of the subject on the basis of the subject data; specifying a data item corresponding to subject data, which contributes to a prediction result of the brain disease, in the subject data; and outputting the specified data item and prior knowledge related to the brain disease in association with each other.

A diagnosis support method according to this embodiment includes: acquiring subject data related to a brain of a subject; predicting a brain disease of the subject on the basis of the subject data; specifying a data item corresponding to subject data, which contributes to a prediction result of the brain disease, in the subject data; and outputting the specified data item and prior knowledge related to the brain disease in association with each other.

It is to be noted that, as used herein and in the appended claims, the singular forms “a”, “an”, and “the” include plural referents unless the context clearly dictates otherwise.
As this invention may be embodied in several forms without departing from the spirit of essential characteristics thereof, the present embodiments are therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1-11. (canceled)

12. A diagnosis support device comprising:
an acquisition unit acquiring subject data including both a feature amount of a medical image related to a brain of a subject and subject information including neuropsychological test information of the subject;
a prediction unit predicting a brain disease of the subject on the basis of the subject data;
a specification unit specifying a data item corresponding to subject data, which contributes to a prediction result of the prediction unit, in the subject data; and
an output unit outputting the data item specified by the specification unit and prior knowledge related to the brain disease in association with each other.

13. A diagnosis support device according to claim 12, further comprising:
a scaling unit scaling the subject data; wherein
the prediction unit predicting a brain disease of the subject on the basis of the scaled subject data.

14. The diagnosis support device according to claim 12,
wherein the acquisition unit acquires, as the subject data, an image feature amount calculated on the basis of the medical image related to the brain of the subject.

15. The diagnosis support device according to claim 14,
wherein the image feature amount includes a degree of atrophy of a part of the brain.

16. The diagnosis support device according to claim 12,
wherein the acquisition unit acquires, as the subject data, subject information including at least one of test information and clinical information related to the brain of the subject.

17. The diagnosis support device according to claim 12, further comprising:
a storage unit storing the prior knowledge as known evidence related to the brain disease in association with a data item related to the brain; and
a contribution degree calculation unit calculating a degree of contribution of the subject data, which contributes to the prediction result,
wherein the specification unit specifies the data item corresponding to the subject data on the basis of the degree of contribution and a predetermined contribution degree threshold value, and
the output unit reads prior knowledge corresponding to the specified data item from the storage unit and outputs the prior knowledge.

18. The diagnosis support device according to claim 12,
wherein the output unit outputs a degree of association between the data item and the prior knowledge.

19. The diagnosis support device according to claim 18, further comprising:
an association degree calculation unit calculating the degree of association on the basis of at least one of the degree of contribution of the subject data corresponding to the data item and a reliability of the prior knowledge as evidence.

20. The diagnosis support device according to claim 12,
wherein the output unit outputs the subject data corresponding to the data item.

21. The diagnosis support device according to claim 12, further comprising:
a display unit displaying the data item, the subject data, and the prior knowledge corresponding to the data item in association with one another in an order of the degree of contribution of the subject data corresponding to the data item to the prediction result.

22. A computer readable non-transitory recording medium recording a computer program causing a computer to execute a process comprising:
acquiring subject data including both a feature amount of a medical image related to a brain of a subject and subject information including neuropsychological test information of the subject;
predicting a brain disease of the subject on the basis of the subject data;
specifying a data item corresponding to subject data, which contributes to a prediction result of the brain disease, in the subject data; and
outputting the specified data item and prior knowledge related to the brain disease in association with each other.

23. A computer readable non-transitory recording medium according to claim 22, recording a computer program causing a computer to execute a process comprising:
scaling the subject data;
predicting a brain disease of the subject on the basis of the scaled subject data.

24. A diagnosis support method comprising:
acquiring subject data including both a feature amount of a medical image related to a brain of a subject and subject information including neuropsychological test information of the subject;
predicting a brain disease of the subject on the basis of the subject data;
specifying a data item corresponding to subject data, which contributes to a prediction result of the brain disease, in the subject data; and
outputting the specified data item and prior knowledge related to the brain disease in association with each other.

25. A diagnosis support method according to claim 24, further comprising:
scaling the subject data;
predicting a brain disease of the subject on the basis of the scaled subject data.
